(19) United States
(12) Patent Application Publication
(10) Pub. No.: US 2025/0339115 A1
(43) Pub. Date: Nov. 6, 2025

(54) Medical Imaging System And Methods

(71) Applicant: Mobius Imaging, LLC, Shirley, MA (US)

(72) Inventor: Robert Coughlin Powell, Stow, MA (US)

(73) Assignee: Mobius Imaging, LLC, Shirley, MA (US)

(21) Appl. No.: 18/868,932

(22) PCT Filed: May 23, 2023
(86) PCT No.: PCT/US2023/023195

§ 371 Date: Nov. 25, 2024

Related U.S. Application Data
(60) Provisional application No. 63/345,504, filed on May 25, 2022.

(51) Int. Cl. A61B 6/06; A61B 6/00; A61B 6/03

(52) U.S. Cl. CPC A61B 6/06; A61B 6/035; A61B 6/542

(57) ABSTRACT

An imaging system having a gantry connected with a base. The gantry includes an x-ray source to produce an x-ray beam, an x-ray detector to receive the x-ray beam from the x-ray source, and an adjustable collimator including a limiter disposed between the x-ray source and the x-ray detector. The limiter is arranged for movement between: a first position to at least partially limit transmission of the x-ray beam towards the x-ray detector according to a first transmission parameter, and a second position to at least partially limit transmission of the x-ray beam towards the x-ray detector according to a second transmission parameter different from the first transmission parameter. A controller of a control system is configured to move the limiter of the adjustable collimator between the first position and the second position to adjust transmission of the x-ray beam from the x-ray source towards the x-ray detector.

### CROSS-REFERENCE TO RELATED APPLICATION

The subject patent application claims priority to and all the benefits of U.S. Provisional Patent Application No. 63/345,504 filed on May 25, 2022, the disclosure of which is hereby incorporated by reference in its entirety.

### BACKGROUND

Conventional medical imaging devices, such as computed tomography (CT) and magnetic resonance (MR) imaging devices, are typically realized with fixed or otherwise relatively immobile devices located in a discrete area reserved for imaging that is often far removed from the point-of-care where the devices could be most useful.

For certain procedures, patient-specific imaging data may be acquired intraoperatively using one or more types of imaging systems to help assist the surgeon in visualizing, navigating relative to, and/or treating the anatomy. To this end, navigation systems may cooperate with imaging systems and/or other parts of surgical systems (e.g., surgical tools, instruments, surgical robots, and the like) to track objects relative to a target site of the anatomy.

Computed tomography imaging systems generally use some form of collimation to reduce the extraneous x-rays that are not used to create the image and prevent unnecessary extra dose to the patient. In many cases, these collimators are static in relation to the X-ray source or can be re-sized similar to a camera aperture. Static collimators provide the same beam size in any scan protocol.

In some examples, the x-ray source and x-ray detector are rotated during a helical scan used for creating a three-dimensional image of a specific area of a patient. In other examples, a scout scan in which the x-ray source and x-ray detector are rotationally stationary may be used to locate a target area within in the patient or confirm placement of a surgical device. The scout scan may only require a fraction of the x-ray intensity that a full helical scan requires. As such, it may be desirable to have an imaging system with a way to adjust the amount of x-ray intensity that is passed from the x-ray source to the x-ray detector.

### SUMMARY

The present teachings generally provide for an imaging system comprising a base, a gantry connected with the base, and a control system. The gantry includes an x-ray source to produce an x-ray beam, an x-ray detector to receive the x-ray beam from the x-ray source, an adjustable collimator including a limiter disposed between the x-ray source and the x-ray detector. The limiter is arranged for movement between a first position to at least partially limit transmission of the x-ray beam towards the x-ray detector according to a first transmission parameter, and a second position to at least partially limit transmission of the x-ray beam towards the x-ray detector according to a second transmission parameter different from the first transmission parameter. The control system includes a controller configured to move the limiter of the adjustable collimator between the first position and the second position to adjust transmission of the x-ray beam from the x-ray source towards the x-ray detector.

The teachings further provide for a method of adjusting a transmission parameter of an imaging system. The imaging system comprises a base, a gantry connected with the base, and a control system. The gantry includes an x-ray source to produce an x-ray beam, an x-ray detector to receive the x-ray beam from the x-ray source, an adjustable collimator including a limiter disposed between the x-ray source and the x-ray detector and arranged for movement between a first position and a second position. The control system includes a controller configured to move the limiter of the adjustable collimator between the first position and the second position. The method comprises controlling the control system to select a scan mode of the imaging system; moving the limiter to a position corresponding with the scan mode of the imaging system; and sending an x-ray beam between the x-ray source and the x-ray detector, passing through the limiter. The limiter in the first position at least partially limits transmission of the x-ray beam towards the x-ray detector according to a first transmission parameter, and the second position of the limiter at least partially limits transmission of the x-ray beam towards the x-ray detector according to a second transmission parameter different from the first transmission parameter.

### BRIEF DESCRIPTION OF THE DRAWINGS

Advantages of the present disclosure will be readily appreciated as the same becomes better understood by reference to the following detailed description when considered in connection with the accompanying drawings.

FIG. 1 is a perspective view of an imaging system.

FIG. 2 is a cross-sectional schematic illustration of an imaging system that illustrates the rotating and non-rotating portions of the system.

FIG. 3 is an enhanced schematic view of a portion of FIG. 2.

FIG. 4 illustrates a drive mechanism of the imaging device.

FIG. 5 illustrates the imaging system performing a helical scan.

FIGS. 6A and 6B show the imaging system moving between a first position and a second position.

FIG. 7 illustrates one example of an x-ray source with a stationary collimator assembly.

FIG. 8 is a perspective view of an adjustable collimator assembly.

FIG. 9 is an exploded view of the adjustable collimator assembly.

FIG. 10 is an exploded view of the adjustable collimator assembly and x-ray source.

FIG. 11 illustrates the adjustable collimator assembly in a first position.

FIG. 12 illustrates the adjustable collimator assembly in a second position different than the first position.

FIG. 13 illustrates the adjustable collimator assembly in a first position.

FIG. 14 illustrates the adjustable collimator assembly in a second position.

FIG. 15 illustrates the adjustable collimator in a third position.

### DETAILED DESCRIPTION

The various versions of the present disclosure will be described in detail with reference to the accompanying drawings. Wherever possible, the same reference numbers will be used throughout the drawings to refer to the same or corresponding parts. References made to particular examples and implementations are for illustrative purposes and are not intended to limit the scope of the present disclosure.

The present disclosure generally relates to an imaging system 100 (also known as a surgical imaging system). The imaging system 100 may be used for pre-operative planning, intraoperative use, and/or post-operative follow up. The imaging system 100 may function with an x-ray imaging device 10 (and/or other types of imaging devices) to acquire x-ray images (e.g., patient imaging data) of one or more anatomical objects of interest and display the x-ray images to a surgeon or surgery team. For example, the imaging system 100 may take and display an x-ray image of a particular patient P anatomical feature or region (e.g., knee, spine, ankle, foot, neck, hip, arm, leg, rib cage, hand, shoulder, head, the like, and/or combinations thereof). In some examples, the imaging system 100 may function to superimpose an image of surgical instruments 106, 108 over the displayed x-ray image of the anatomical feature, displaying the surgical instruments 106, 108 relative the anatomical feature. The imaging system 100 may function to acquire multiple x-ray images forming a CT scan of a patient P. The imaging system 100 may be configured to automatically correlate a position of an x-ray imaging device 10 with a portion of the x-ray images taken during a scan. The imaging system 100 may register the x-ray images with the position of the x-ray images based on information generated by the navigation system 16 including an optical sensor (e.g., camera units 56 of a localizer 54). In some versions, the imaging system 100 comprises an x-ray imaging device 10 (also referred to as an imager) including a base 20, a gimbal 30, a gantry 40, and a pedestal 50. The gantry 40 is configured to translate along the base 20.

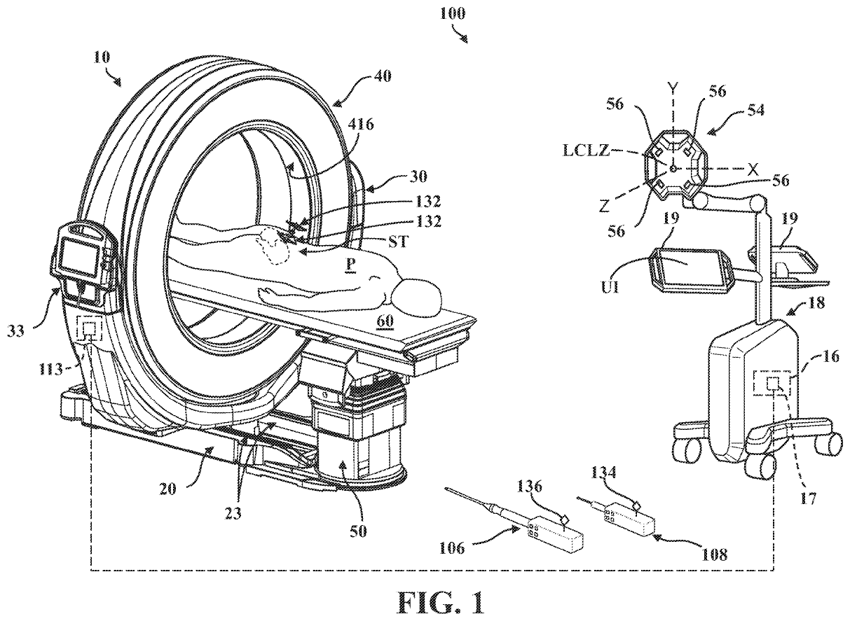

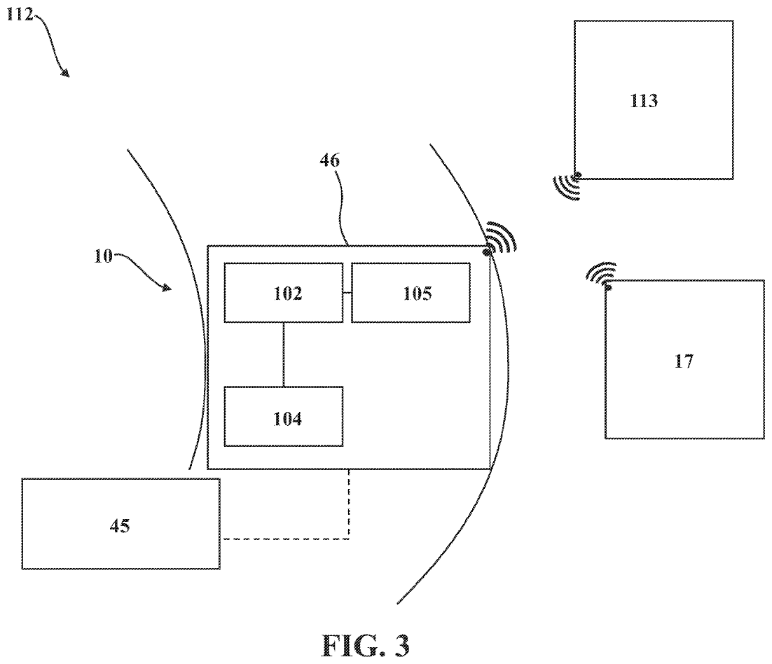

Referring to FIG. 1, in some versions, the navigation system 16 may employ a navigation controller 17 that communicates with an imager system controller 113 of the x-ray imaging device 10. The imaging system 100 is configured to collect imaging data, such as, for example x-ray computed tomography (CT) or magnetic resonance imaging (MRI) data, from an object located within a bore 416 of the gantry 40, in any manner known in the medical imaging field, and to register the collected imaging data in a navigation reference frame of the navigation system 16. As best seen schematically in FIG. 3, at least the imager system controller 113, the navigation controller 17, a controller 46 (also referred to as an “on board computer”) may for part of the control system 112 of the imaging system 100 as described in greater detail below.

Referring to FIG. 1, as noted above, the imaging system 100 may include the navigation system 16. One example of the navigation system 16 is described in U.S. Pat. No. 9,008,757 filed on Sep. 24, 2013, the entire disclosure of which is hereby incorporated by reference. The navigation system 16 tracks movement of various objects, such as, for example, portions of the x-ray imaging device 10 (e.g., gantry 40, rotor 41, base 20, pedestal 50, tabletop support 60), one or more surgical instruments 106, 108 or tools, anatomy of a patient P (e.g., the spine or other bone structures, such as one or more vertebra, the pelvis, scapula, or humerus), and/or combinations thereof. The navigation system 16 monitors or otherwise tracks these objects and may gather state information of each object with respect to a (navigation) localizer coordinate system LCLZ. As used herein, the state of an object includes, but is not limited to, data that defines the position and/or orientation of the tracked object (e.g., coordinate systems thereof) or equivalents/derivatives of the position and/or orientation. For example, the state may be a pose of the object, and/or may include linear velocity data, angular velocity data, and the like. In some examples, such as shown in FIG. 1, the navigation controller 17 is operatively connected with the control system 112 of the imaging system 100.

The navigation system 16 may employ a mobile cart assembly 18 that houses a navigation controller 17, and/or other types of control units. A navigation user interface UI is in operative communication with the navigation controller 17. The navigation user interface UI includes one or more display devices 19. The navigation system 16 is capable of displaying graphical representations of the relative states of the tracked objects to the user using the one or more display devices 19. The navigation user interface UI further comprises one or more input devices (not shown in detail) to input information into the navigation controller 17 or otherwise to select/control certain aspects of the navigation controller 17. Such input devices include interactive touchscreen displays. However, the input devices may include any one or more of push buttons, pointer, foot switches, a keyboard, a mouse, a microphone (voice-activation), gesture control devices, and the like. In some examples, the user may use buttons located on the surgical instrument 106 (e.g., a pointer) to navigate through icons and menus of the user interfaces UI to make selections, configuring the imaging system 100 and/or advancing through the workflow.

In the illustrated versions, the localizer 54 of the navigation system 16 is coupled to the navigation controller 17. In some versions, the localizer 54 is an optical localizer and includes a camera unit 56. In certain configurations, the localizer 54 may be similar to as is described in U.S. Pat. No. 10,959,783 filed Apr. 15, 2016, the entire disclosure of which is hereby incorporated by reference. The localizer 54 may function to monitor and track tracking devices 132, 134, 136 (also referred to as “trackers”) that are coupled to or otherwise supported on various tracked objects, such as the x-ray imaging device 10, surgical instruments 106, 108, the patient P, and/or combinations thereof. One suitable localizer 54 is the FP8000 tracking camera manufactured by Stryker Corporation (Kalamazoo, Mich.).

As best shown in FIG. 1, the pedestal 50 is adapted to support a tabletop support 60 that can be attached to the pedestal 50 in a cantilevered manner and extend out into the bore 416 of the gantry 40 to support a patient P or other object being imaged. In some examples, the tabletop support 60 can be partially or entirely removed from the pedestal 50, and the gantry 40 can be rotated relative to the base 20, preferably at least about 90 degrees, from an imaging position to a transport position to facilitate transport and/or storage of the x-ray imaging device 10.

The x-ray imaging device 10 functions to acquire images of the patient P or anatomical features of the patient's P body supported on the tabletop support 60 (or on some other type of patient support). The x-ray imaging device 10 may include a structure with an emitting portion realized as an x-ray source 43 (e.g., one or more x-ray tubes or other types of radiation sources) and an imaging portion realized as an x-ray detector 34 (or some other form of detector). The x-ray imaging device 10 may be configured to have a gantry 40 with a general O-shape. The gantry 40 may include the x-ray source 43 and the x-ray detector 45 located on the opposing portions of the gantry 40. The x-ray source 43 and the x-ray detector 45 may be at a fixed distance from each other. An imaging region (not shown in detail) may be defined in the center of the O-shape, within the bore 416, between the x-ray source 43 and the x-ray detector 45. A patient P or a portion of a patient P may be located in the center of the bore 416 of the gantry 40, between the x-ray source 43 and the x-ray detector 45, so that a specific portion of the patient P may be imaged.

The outer diameter of the gantry 40 can be relatively small, which may facilitate the portability of the x-ray imaging device 10. In one example, the outer diameter of the gantry 40 is less than about 70 inches, such as between about 60 and 68 inches, and in some versions is about 66 inches. The outer circumferential wall of the outer shell 42 may be relatively thin to minimize the outer diameter dimension of the gantry 40. In addition, the interior diameter of the gantry 40, or equivalently the bore 416 diameter, can be sufficiently large to allow for the widest variety of imaging applications, including enabling different patient supports 60 (e.g., tabletop supports 60) to fit inside the bore 416, and to maximize access to a subject located inside the bore 416. In some versions, the bore diameter of the gantry 40 is greater than about 38 inches, such as between about 38 and 44 inches, and in some versions can be between about 40 and 50 inches. In one exemplary version, the bore 416 has a diameter of about 42 inches. The gantry 40 generally has a narrow profile, which may facilitate portability of the x-ray imaging device 10. In some versions, the width of the gantry 40 is less than about 17 inches and can be about 15 inches or less.

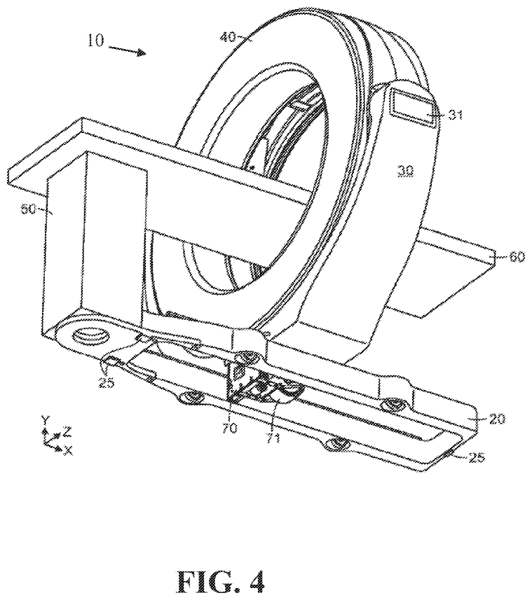

As is best depicted in FIG. 4, the x-ray imaging device 10 includes a drive mechanism 70 mounted beneath the gimbal 30 and the gantry 40 and within the base 20. The drive mechanism 70 also comprises a drive wheel 71 that can extend and retract between a first extended position to facilitate transport of the x-ray imaging device 10, and a second retracted position during an image acquisition procedure (e.g., during an imaging scan). The drive mechanism 70 includes a main drive (not shown in detail) that is geared into the drive wheel 71 when the drive wheel 71 is in the first extended position to propel the x-ray imaging device 10 across a floor or other surface, and thus facilitate transport and positioning of the x-ray imaging device 10. In some versions, the drive wheel 71 can be decoupled from the main drive when the drive wheel 71 is in the second retracted position, thus preventing the x-ray imaging device 10 from back-driving the main drive during an imaging procedure. In some versions, the drive mechanism 70 includes one or more sensors (not shown) to track the position of the drive wheel 7, the position of the gimbal 30 and gantry 40, and the like, relative to the base 20 and/or to other components of the x-ray imaging device 10.

As is illustrated in FIG. 1, the base 20 is realized as a sturdy, generally rectilinear support structure, and includes a central opening extending lengthwise along the base 20 in which the drive mechanism 70 is positioned. In some examples the bottom of the base 20 includes a plurality of pockets (not shown in detail) that contain casters 21 that are retractable. The casters 21 can be spring-loaded and biased to extend from the bottom of the base 20 when the x-ray imaging device 10 is raised off the ground. When the drive wheel 71 is retracted and the x-ray imaging device 10 is lowered to the ground, the casters 21 are retracted into their respective pockets. In an alternative version, an active drive system, rather than a passive spring-based system, can drive the extension and retraction of the casters in their respective pockets.

The gimbal 30 may be a generally C-shaped support that is mounted to the top surface of base 20 and includes a pair of arms 31, 33 extending up from the base. The arms 31, 33 may be connected to opposite sides of gantry 40 so that the gantry is suspended above base 20 and gimbal 30. In some versions, the gimbal 30 and gantry 40 may rotate together about a first (e.g., vertical) axis with respect to the base 20, and the gantry 40 may tilt about a second (e.g., horizontal) axis with respect to the gimbal 30 and base 20. In some versions, a gimbal drive mechanism (not shown in detail) may be mounted between the gimbal 30 and the base 20 to controllably drive the rotation (i.e., “yaw” motion) of the gimbal 30 and gantry 40 with respect to the base 20. A gimbal drive mechanism may also controllably drive the “tilt” motion of the gantry 40 with respect to the gimbal 30.

The gimbal 30 and gantry 40 may translate with respect to the base 20. The gimbal 30 may include bearing surfaces (not shown in detail) that travel on rails 23, as shown in FIG. 1, to provide the translation motion of the gimbal 30 and gantry 40. A scan drive mechanism (not shown in detail) may drive the translation of the gantry 40 and gimbal 30 relative to the base 20, and a main drive mechanism may drive the entire system in a transport mode (e.g., on one or more casters or wheels). In the version of FIG. 1, both of these functions are combined in the drive mechanism 70 that is located beneath the gimbal 30. Further details of similar drive mechanisms 70 for x-ray imaging devices 10 are described in U.S. Pat. No. 8,753,009 filed Feb. 11, 2011, the entire disclosure of which is hereby incorporated by reference.

The x-ray imaging device 10 generally operates to obtain images of an object located in the bore 416 of the gantry 40. For example, in the case of an x-ray CT scan, the rotor 41 rotates within the housing of the gantry 40 while imaging components, including the x-ray source 43 and x-ray detector 45, obtain image data at a variety of scan angles. Generally, the x-ray imaging device 10 obtains image data over relatively short intervals, with a typical scan lasting less than a minute, or sometimes just a few seconds. During these short intervals, however, a number of components, such as the x-ray source 43 and the high-voltage generator 44, require a large amount of power, including, in some versions, up to 32 kW of power.

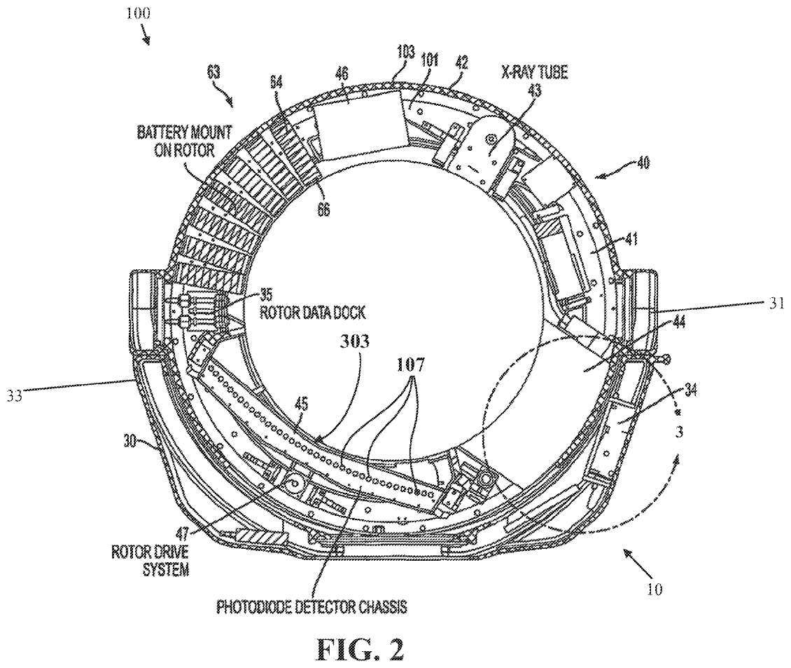

The example illustrated in FIG. 2 illustrates a single high voltage generator 44 powering the x-ray source 43. However, it will be understood that in various versions multiple high voltage generators 44 may be provided on the gantry 40, and each x-ray source 43 may have a dedicated high-voltage generator 44. In some versions, one or more high-voltage generators 44 may be provided off of the gantry 40, and high voltage power may be delivered to the x-ray source 43 via a cable or slip ring system (not shown).

The high-voltage generator 44 may be powered by a power source on the gantry 40, such as a battery system 63. As shown in FIG. 2, the battery system 63 may be mounted to and rotates with the rotor 41. The battery system 63 may include a plurality of electrochemical cells. The cells may be incorporated into one or more battery packs. The battery system 63 is preferably rechargeable and may be recharged by a charging system (not shown) between imaging operations, such as when the rotor 41 is not rotating. In some versions, the battery system 63 consists of lithium iron phosphate (LiFePO4) cells, though it will be understood that other suitable types of batteries can be utilized.

The battery system 63 provides power to various components of the x-ray imaging device 10. In particular, since the battery system 63 is located on the rotor 41, the battery system 63 may provide power to any component on the rotor 41, even as these components are rotating with respect to the non-rotating portion of the x-ray imaging device 10. Specifically, the battery system 63 is configured to provide the voltages and peak power required by the high-voltage generator 44 and x-ray source 43 (e.g., the x-ray tube) to perform an imaging scan. For example, a battery system 63 may output ˜360V or more, which may be stepped up to 120 kV at the high-voltage generator 44 to perform an imaging scan. In addition, the battery system 63 may provide power to operate other components, such as an on-board computer 46, the x-ray detector 45, and a drive mechanism 47 for rotating the rotor 41 within the gantry 40. Here, in some versions, the drive mechanism 47 drives the rotation of the rotor 41 around the interior of the gantry 40. The drive mechanism 47 may be controlled by the imager system controller 113 that controls the rotation and precise angular position of the rotor 41 with respect to the gantry 40, such as by using position feedback data from one or more encoder devices (not shown). The drive mechanism 47 may include a motor and gear system mounted to the rotor 41 (see FIG. 2; not shown in detail). The motor may drive a gear that may be engage with a mating component on the non-rotating portion of the x-ray imaging device 10 to drive the rotation of the rotor 41. For example, a belt 82 may be rotatably fixed on the non-rotating portion of the x-ray imaging device 10 (e.g., the outer shell of the gantry 40), such as on a circumferential rail. The drive mechanism 47 may engage with the belt 82 to drive the rotation of the rotor 41 within the gantry 40. The drive mechanism 47 may be powered by the battery system 63, may be secured to the rotor 41, and may be positioned behind the x-ray detector 45, as shown in FIG. 2. Further details of a similar type of drive mechanisms 47 are described in U.S. Pat. No. 9,737,273 filed Apr. 6, 2012, the entire disclosure of which is hereby incorporated by reference.

An on-board computer 46 may be provided on the rotating portion of the system and may be secured to rotor 41 in a suitable location, as shown in FIG. 2. FIG. 3 is an enhanced schematic view of the on-board computer 46 including processor 102, memory 104, and transmitter/receiver 105. The on-board computer 46 may be connected with one or more external computers and/or controllers 113 of the control system 112 in a wired or wireless link. The on-board computer 46 may be powered by battery system 63. The on-board computer 46 may be any suitable computing device, and may include one or more processors 102 having associated memory 104 that may execute instructions (e.g., software) stored in memory 104, as is known in the art. The on-board computer 46 may perform various control functions for the various components on the rotor 41 and may serve as an interface between components on the rotor 41 and other components of the x-ray imaging device 10. The on-board computer 46 may be configured to receive imaging data collected by the x-ray detector 45. For example, the x-ray detector 45 may stream their image data over a suitable data connection (e.g., wired or wireless) to the on-board computer 46. The on-board computer 46 may store, process and/or transmit the imaging data. For example, the on-board computer 46 may include or may be coupled to a wireless transmitter that may transmit the data to another logical entity, such as to an external workstation and/or to another controller 113 located on the non-rotating portion of the system (e.g., in the gimbal 30). This may enable real-time display of the collected imaging data.

A docking system 35 may be provided for connecting the rotating portion of the x-ray imaging device 10 to the non-rotating portion between imaging scans. The docking system 35 may include a connector for carrying power between the rotating and non-rotating portions. In some versions, the docking system 35 may be used to provide power to the battery system 63 such that the batteries may be charged using power from an external power source (e.g., grid power). The docking system 35 may also include a data connection to allow data signals to pass between the rotating and non-rotating portions. Further details of a suitable docking system are described in U.S. Pat. No. 9,737,273 filed Apr. 6, 2012, the entire disclosure of which is hereby incorporated by reference.

During an imaging scan, the rotor 41 rotates around an object positioned within the bore 416, while the imaging components such as the x-ray source 43 and x-ray detector 45 operate to obtain imaging data (e.g., raw x-ray projection data) for an object positioned within the bore 416 of the gantry 40, as is known, for example, in conventional X-ray CT scanners. The collected imaging data may be fed to an on-board computer 46, preferably as the rotor 41 is rotating, for performing x-ray CT reconstruction, as will be described in further detail below.

Various details of examples of an imaging system can be found in the above-referenced U.S. Pat. No. 8,118,488 filed Jan. 5, 2009, U.S. Pat. No. 8,753,009 filed Mar. 9, 2010, U.S. Pat. No. 8,770,839 filed Mar. 19, 2010, and U.S. Pat. No. 9,737,273 filed Apr. 7, 2011, which have been incorporated herein by reference. It will be understood that these examples are provided as illustrative, non-limiting examples of imaging systems suitable for use in the present methods and systems, and that the present systems and methods may be applicable to imaging systems of various types, now known or later developed.

The x-ray detector 45 may include a plurality of x-ray sensitive detector elements, along with associated electronics, which may be enclosed in a housing or detector chassis 303 (FIG. 2). In one example, the detector chassis has a width of 7¾ inches, a depth of between about 4-5 inches and a length of about 1 meter or more, such as about 43 inches. The detector chassis 303 may be a rigid frame, which may be formed of a metal material, such as aluminum, and which may be formed by a suitable machining technique. The x-ray detector 45 may be mounted to the rotor 41 opposite an x-ray source 43, as is shown in FIG. 2. A plurality of x-ray-sensitive detector elements are located in within detector modules 107 provided in the interior of the detector chassis 303 so that the detector elements face in the direction of the x-ray source 43. The detector chassis 303 may form a protective air- and light-tight shroud around the detector elements, so that unwanted air and light may not contaminate the sensitive components housed within the x-ray detector 45.

In various examples, the individual detector elements may be located on a plurality of detector modules 107. FIG. 3 illustrates a plurality of detector modules 107 arranged within a detector chassis 303 of x-ray detector 45. Each individual detector element, which may be for example, a cadmium tungstate (CdWO₄) material coupled to a photodiode, represents a pixel on a detector module 107 with multiple elements. The detector modules 107 may be 2D element array, with for example 512 pixels per module (e.g., 32×16 pixels).

The x-ray detector 45 may include one or more detector modules 107 mounted within the detector chassis 303. The detector module(s) 107 may be arranged along the length of the detector chassis 303 to form or approximate a semicircular arc, with the arc center coinciding with the focal spot of detector the x-ray source 43. In one example, the x-ray detector 45 includes thirty-one two-dimensional detector modules 107 positioned along the length of the detector chassis 303. and angled relative to each other to approximate a semicircular arc centered on the focal spot of the x-ray source. Each detector module 107 may be positioned such that the detector module 107 surface is normal to a ray extending from the x-ray focal spot to the center pixel of the detector module 107.

It will be understood that the x-ray detector 45 may include any number of detector modules 107 along the length of the detector. As shown in FIG. 3, for example, a detector may include “m” modules 107, where “m” may be any integer greater than or equal to 1. Further, each detector module 107 may include an arbitrary number of individual elements (pixels) in the module. Larger and/or a greater number of detector modules 107 may allow a larger diameter “back projection” area around the isocenter of the imaging system, and thus may allow a larger cross-section of the object to be reconstructed.

Each of the detector modules 107 may include an array of photosensitive elements which may be electrically and optionally physically coupled to a circuit board that may include one or more electronic components. In some examples, the detector modules 107 may plug into a circuit board using a suitable electronic connection such as described in U.S. Pat. No. 9,111,379 filed Jun. 28, 2012, which is incorporated herein by reference in its entirety. The circuit board may be configured to couple the raw analog signals from each detector element in the array into an analog-to-digital converter (herein referred to as A/D converter) for converting the signal to a digital signal. In some examples, the circuit board includes several A/D converters. Each detector element may provide its analog signal over a separate channel into the A/D converters. For example, where the array includes 512 pixels, four 128-channel A/D converters may be provided to convert the analog signal from each element into a digital signal.

The circuit board may include a processor, which may be, for example, an FPGA. The processor may receive the digital image data from the A/D converters, which may be in a digital video format, such as LVDS, and may be programmed to assemble the data into a single image. The processor may be configured to convert the image data to a different digital video format, such as Camera Link. In examples, the processor may convert the image data into another suitable format, such as gigabit Ethernet. The processor may also be programmed to receive image data from one or more other detector modules 107, which may be combined with the image data from the A/D converter(s) and passed off of the detector module 107 in a daisy-chain configuration. In some examples, the processor may receive and transmit the image data in a Camera Link digital video format.

It will be understood that the number of modules (m) in the x-ray detector 45 may vary, and modules may be added or removed as needed. In various examples, changing the number and/or types of detector modules does not require a new or modified “backplane” electronics board, for example. Also, the clock signal (e.g., a Camera Link clock signal) may be variable to provide more or less image frames per second.

As shown in the examples of FIGS. 2 and 3, the detector modules 107 of the x-ray detector 45 may be electronically connected to the on-board computer 46 which may be located on the rotatable portion 101 of the system (e.g., mounted to the rotor 41). The processor 102 of the on-board computer 46 may be configured to perform tomographic reconstruction of image data that is sent to the on-board computer 46 from the detector modules 107. The on-board computer 46 may wirelessly transmit tomographic reconstruction data (e.g., 3D images of the object) to the imager system controller 113, which may be another computer, such as an external workstation, or a separate computer on the imaging system 100 (e.g., a computer on a gimbal that supports the gantry). In other examples, the on-board computer 46 may transmit tomographic reconstruction data to another entity using a wired link (e.g., via a slip ring or cable connection to the non-rotating portion 103, or via a data dock to the non-rotating portion 103 in between scans). In some examples, it will be understood that in addition to on-board computer 46 and x-ray detector 45, the processor 102 for performing the reconstruction may be at any location on the rotating portion 101 (e.g., rotor 41).

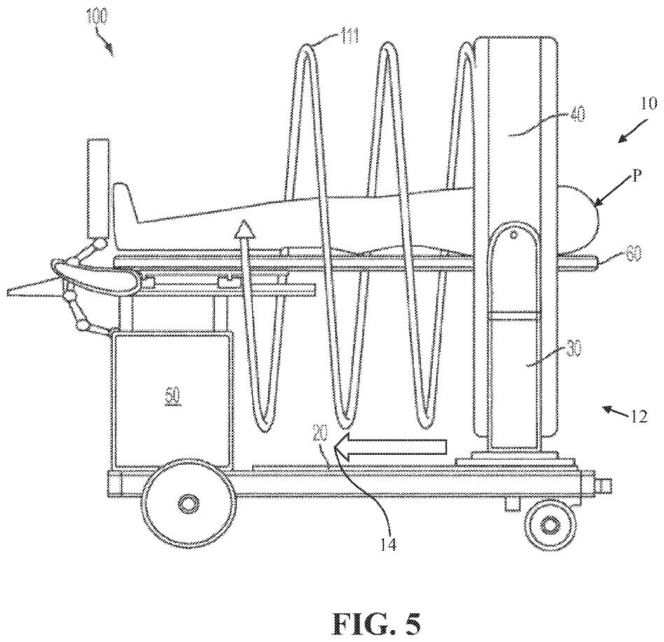

The imaging system 100 may be used to perform cone beam CT imaging. The rotor 41 may rotate within the gantry 40 while the x-ray detector 45 obtain images. The image data may then be reconstructed using a tomographic algorithm as is known in the art to obtain a 3D reconstructed image of the object. In some examples, the x-ray detector 45 may obtain images which may be combined for the reconstruction. FIG. 5 illustrates an example helical scan path of the gantry 40 and the rotation of the x-ray source 43 and x-ray detector 45 on rotor 41 between a first position 12 and a second position 14. In some examples, the rotor 41 may only need to rotate a portion of the distance that would normally be required (e.g., a 90° rotation of the rotor 41 may enable the detector to scan 180° of the object, a 270° rotation of the rotor 41 enables a full 360° scan of the object). In some versions, the gantry 40 and gimbal 30 may be translated along rails 23 during cone beam CT imaging to provide a helical cone beam CT scan (FIG. 5). In some versions, a helical cone beam scan may be coordinated with the injection of a contrast agent to provide a three-dimensional arterial roadmap image.

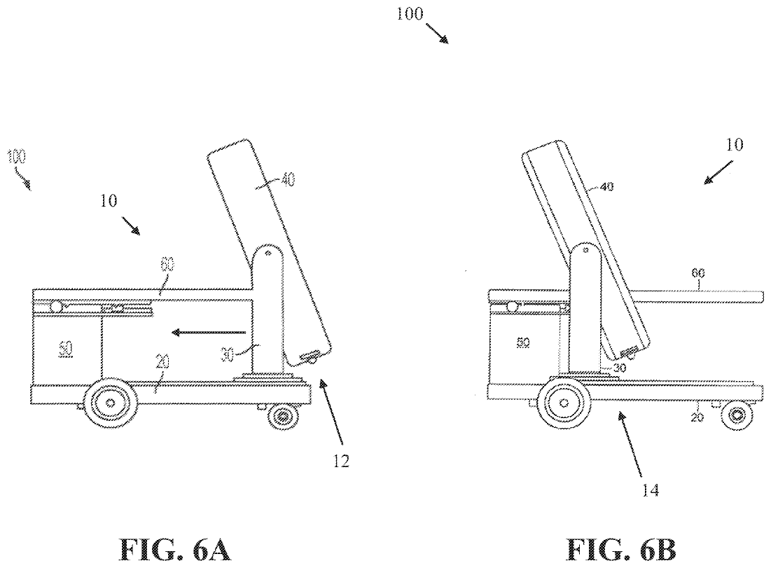

As mentioned above, the gantry 40 may be moved between a plurality of positions and is configured to translate and/or tilt about the base 20 of the x-ray imaging device 10. The gantry 40 is configured to move relative the base 20 to capture x-ray images of a patient P or anatomical feature of interest (e.g., a target site ST), at one or more angled relative to a patient P or particular anatomical feature, raise, lower, repositioned, or a combination thereof. During movement, the x-ray source 43 and the x-ray detector 45 maintain a fixed relationship, keeping the same distance on the opposite ends of the gantry 40. As best seen in FIGS. 6A and 6B, the gantry 40 is configured to move between a first position 12 and second position 14 and may include a plurality of intermediate positions (e.g., transistor and/or intermittent movement) between the first position 12 and the second position 14.

In various examples, the imaging system 100 may be used to pass “scout” scan data from the rotor 41 in real-time. FIGS. 6A and 6B illustrate the gantry 40 translating along the base 20 between positions 12, 14. In FIG. 6A, the gantry 40 is in a first position 12 and FIG. 6B illustrates the gantry 40 in the second position 14 after the gantry 40 has translated along the base 20. A scout scan may be performed while the rotor 41 is not rotating to provide a series of scan lines of the patient (e.g., as the source and detector translate along the patient axis), which may be useful, for example, in choosing a subregion to perform a full 3D scan. The scan lines may be provided from the x-ray detector 45 to processor 102, as described above, which may transmit the scan lines in real time to an external entity (such as a workstation or other computer) for displaying a 2D image of the patient in real-time. During a scout scan, the x-ray beam from the x-ray source 43 may only require a fraction of the size of the x-ray beam required for a full helical scan since the scout scan a preview of the surgical area.

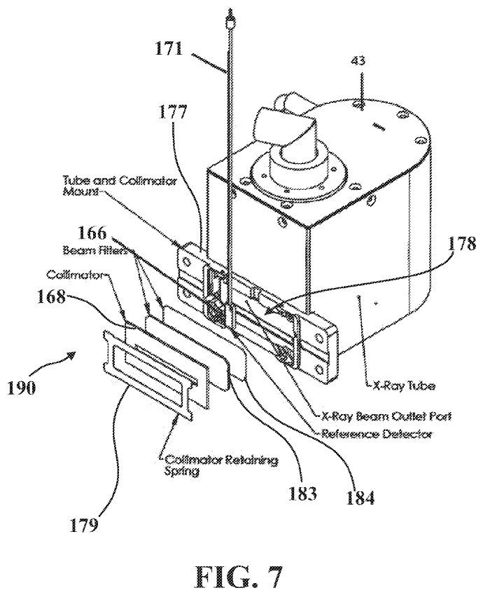

FIG. 7 shows one example of an x-ray source 43 with a non-adjustable collimator assembly 190 with a reference detector 166 and a fiber optic cable 171 assembly. The non-adjustable collimator assembly 190 includes a stationary collimator 168 that is stationary relative to the x-ray source 43. The stationary collimator 168 is connected to a mount 177 locating the stationary collimator 168 axially with the x-ray beam outlet port 178. Beam filters 183, 184 are disposed between the mount 177 and the stationary collimator 168. The stationary collimator 168 and beam filters 183, 184 are held to the mount 177 by a retaining spring 179. In this example, the x-ray beam produced by the x-ray source 43 will fully illuminate the x-ray detector 45 when an image is taken.

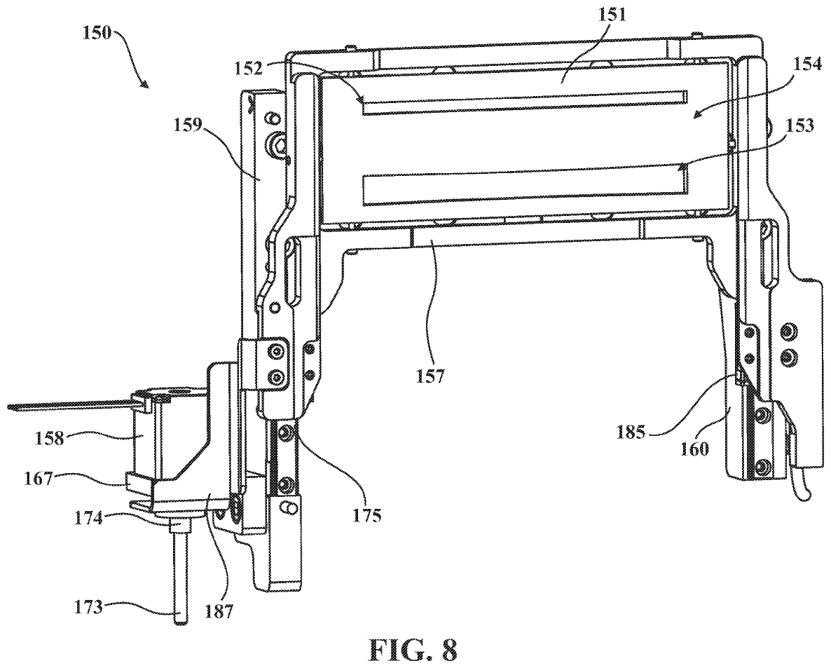

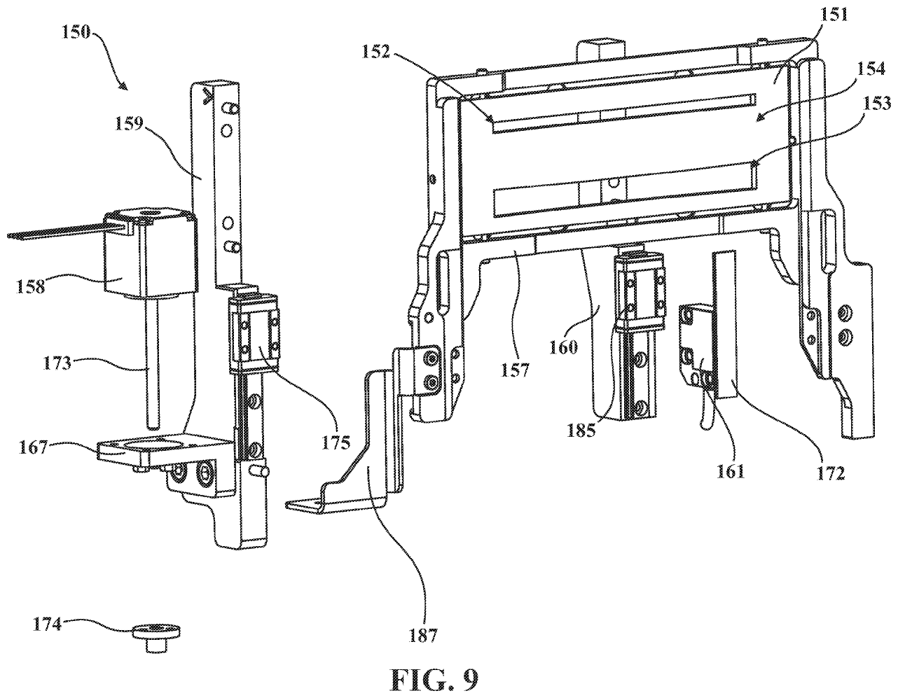

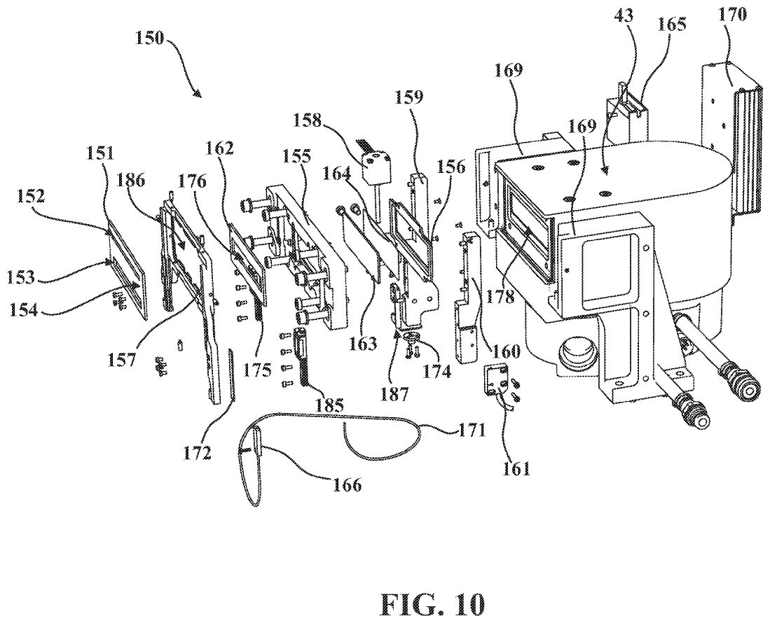

Turning to FIGS. 8-14, the x-ray source 43 is shown with an adjustable collimator assembly 150. FIGS. 8 and 9 illustrate the adjustable collimator assembly 150 removed from the x-ray source 43. FIGS. 9 and 10 illustrate exploded views of the adjustable collimator assembly 150. The adjustable collimator assembly 150 is configured to move between a plurality of positions 180, 181, 182 to alter the amount of x-ray radiation transmission that passes to the x-ray detector 45 depending on the scan mode of the imaging system 100. The position of the adjustable collimator assembly 150 adjusts one or more transmission parameters between positions 180, 181, 182. A transmission parameter is a condition of the x-ray beam relates to the intensity of the x-ray beam between the x-ray source 43 and the x-ray detector 45. In some examples, each of the positions 180, 181, 182 correspond with a transmission parameter.

The adjustable collimator assembly 150 includes a limiter 151. In some examples, portions of the limiter 151 are approximately the size of the x-ray beam outlet port 178. In some examples, the limiter 151 includes one or more apertures (e.g., a first aperture 153, a second aperture 152) for altering the one or more transmission parameters between the x-ray source 43 and the x-ray detector 45. In some examples, the apertures 152, 153 are different sizes for allowing different amounts of the x-ray radiation to pass from through from the x-ray source 43 to the x-ray detector 45. As shown in FIGS. 8-13, the first aperture 153 is larger than the second aperture 152. In some versions, the limiter 151 also includes a blocker 154 (e.g., defined by a solid surface, by the absence of an aperture arranged to permit transmission, and the like) which, when positioned in line with the x-ray beam outlet port 178, does not allow x-ray radiation to pass through the limiter 151 from the x-ray source 43 to the x-ray detector 45.

The adjustable collimator assembly 150 is configured to move the limiter 151 between a plurality of positions (e.g., a first position 182, a second position 181/180, a third position 180/181, and the like). To move the limiter 151, the limiter 151 is coupled to a movable frame 157. The movable frame 157 is configured to receive the limiter 151 in opening 186 of the movable frame 157. The opening 186 is size to accept the limiter 151. The movable frame 157 is in communication with the limiter actuator 158 to move the limiter 151 between the plurality of positions 180, 181, 182.

Turning to FIGS. 8 and 9, the movable frame 157 connects with rails 159, 160 through frame mounts 175, 185. Each of the frame mounts 175, 185 slidable connect the movable frame 157 and limiter 151 with the rails 159, 160 to move the movable frame 157 and the limiter 151 relative to the x-ray beam outlet port 178 when limiter actuator 158 is actuated. A limiter mount 187 connects frame mount 175 to the leadscrew 173 of the limiter actuator 158 and actuator nut 174. The actuator nut 174 is attached to the limiter mount 187 such that the actuator nut 174 does not rotate relative to the limiter mount 187. The leadscrew 173 is disposed through limiter mount 187 into the actuator nut 174 which translates the limiter mount 187 and actuator nut 174 along the leadscrew 173 when the limiter actuator 158 is actuated. When the leadscrew 173 is rotated by the limiter actuator 158, the actuator nut 174 and limiter mount 187 are translated along at least a portion of the length of the leadscrew 173, moving the movable frame 157 and limiter 151 between positions 180, 181, 182. The limiter actuator 158 is connected to rail 159 at the actuator mount 167 which holds the limiter actuator 158 in place. On the other side, position sensor 161 and magnetic strip 172 are disposed along rail 160, adjacent to frame mount 185 on rail 160. In this example, the position sensor 161 is an encoder.

FIG. 10 illustrates an exploded view of the adjustable collimator assembly 150. Positioned between the movable frame 157 and rails 159, 160 is the mount plate 155. The mount plate 155 holds a non-adjustable collimator arrangement comprising beam reducer 162, filters 163, 164, and filter frame 156. Similar to the non-adjustable collimator assembly of FIG. 7, the non-adjust collimator arrangement shown in FIG. 10 is disposed axially with the x-ray beam outlet and functions to filter and focus the x-ray transmission from the x-ray source 43. In some examples, the beam reducer may be made of tungsten. In some examples, filter 163 may be aluminum and filter 164 may be copper. The beam reducer 162 is positioned between the movable frame 157 and the mount plate 155. As described above, the movable frame 157 and limiter 151 are moved between positions 180, 181, 182. Due to size constraints within the imaging system 100, travel is limited. The movable frame 157 and the limiter 151 are configured to move between the plurality of positions 180, 181, 182, however, travel space is limited. Therefore, in order to fit the plurality of positions 180, 181, 182 in the space provided, the beam reducer 162 (also known as a primary collimator) is fixed between the x-ray source 43 and the limiter 151, which reduces the beam size and prevents x-ray scatter from escaping thru the unused apertures or the sides of the limiter 151. Before the x-ray beam enters the beam reducer 162, the x-ray beam passes through filters 163, 164. The filters 163, 164 are held to mount plate 155 by filter frame 156. The mount plate 155 is attached to rails 159, 160, which are connected with collimator mounting arms 169 on the x-ray source 43. The mount plate 155 positions the beam reducer 162 and filters 163, 164 in line with the x-ray beam outlet port 178.

As described above, the limiter actuator 158 is connected with the movable frame 157 through limiter mount 187, moving the limiter 151 through a plurality of positions 180, 181, 182. The limiter actuator 158 is in communication with controller 165 which commands the limiter actuator 158 to rotate leadscrew 173, translating actuator nut 174 and the limiter mount 187 to move the movable frame 157 and limiter 151 between positions 180, 181, 182. Controller 165 is connected with one or more controllers 17, 46, 113 of the control system 112 of the imaging system 100 and is configured to actuate the limiter actuator 158 to move the adjustable collimator assembly 150 between positions 180, 181, 182. In some examples, the controller 165 is configured to automatically actuate the limiter actuator 158 to position the limiter 151 based on the imaging mode selected. In other examples, the controller 165 commands the limiter actuator 158 to actuate the adjustable collimator assembly 150 when a user has selected the desired position 180, 181, 182 of the adjustable collimator assembly 150.

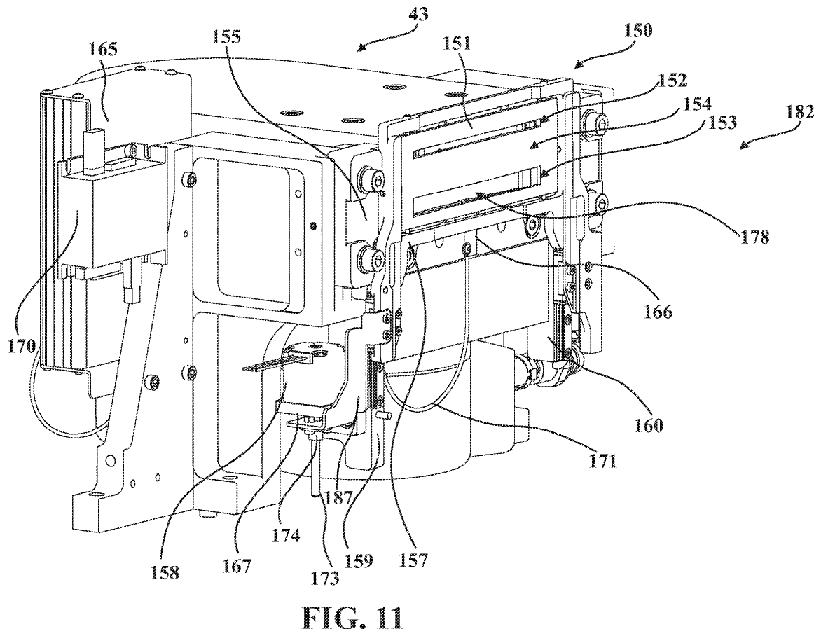

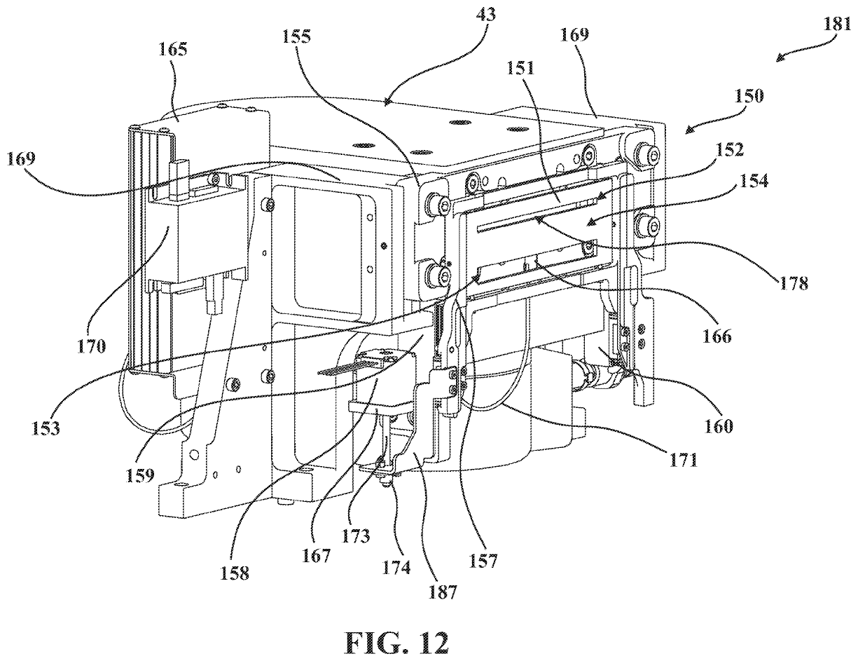

FIGS. 11 and 12 illustrate a side perspective view of adjustable collimator assembly 150 moved to positions 182 and 181. In the example shown in FIG. 11, the adjustable collimator assembly 150 is in position 182, positioning aperture 153 of the limiter 151 in line with the beam reducer 162 and the x-ray beam outlet port 178. The limiter actuator 158 rotates the leadscrew 173 such that the actuator nut 174 and limiter mount 187 are translated toward the limiter actuator 158, moving the movable frame 157 and aperture 153 into alignment with the x-ray beam outlet port 178. In position 182, an x-ray beam will fully illuminate the x-ray detector 45 when an image is taken by the imaging system 100. In some examples, a first transmission parameter, such as a first x-ray beam intensity, corresponds with the alignment of aperture 153 with the x-ray beam outlet port 178 in position 182. In this position, the imaging system 100 may be in a CT scan mode where a helical scan is performed, allowing the x-ray transmission to pass from the x-ray source 43 through the window 176 of the beam reducer 162 and limiter 151 to the x-ray detector 45 without reducing the beam size between the beam reducer 162 and the x-ray detector 45. The x-ray beam from the x-ray source 43 will maintain beam size between the beam reducer 162 and the x-ray detector 45 because aperture 153 of the limiter 151 and the window 176 of the beam reducer 162 are substantially similar in size.

Turning to FIG. 12, the adjustable collimator assembly 150 is in position 181, aligning aperture 152 with the beam reducer 162 and the x-ray beam outlet port 178. The limiter actuator 158 rotates the leadscrew 173 such that the actuator nut 174 and limiter mount 187 are translated away from the limiter actuator 158, moving the movable frame 157 and aperture 152 into alignment with the x-ray beam outlet port 178. In position 181, an x-ray beam will partially illuminate the x-ray detector 45 because aperture 152 is smaller than the window 176 of the beam reducer 162, reducing the x-ray beam size which passes from the x-ray source 43 to the x-ray detector 45. In some examples, a second transmission parameter, such as a second x-ray beam intensity that is less than the first x-ray beam intensity, corresponds with the alignment of aperture 152 with the x-ray beam outlet port 178 in position 181. In this position, the imaging system 100 may be in a scout scan mode, reducing the x-ray transmission passing from the x-ray source 43 through the limiter 151 to the x-ray detector 45. The x-ray beam from the x-ray source 43 will be reduced in beam size as the x-ray beam passes through aperture 152 of the limiter 151, since aperture 152 is more narrow than the window 176 of the beam reducer 162. Since the aperture 152 of the limiter 151 is more narrow than the window 176 of the beam reducer 162, the x-ray beam from the x-ray source is partially blocked from passing to the x-ray detector 45 reducing the exposure of the x-ray beam on a patient being imaged.

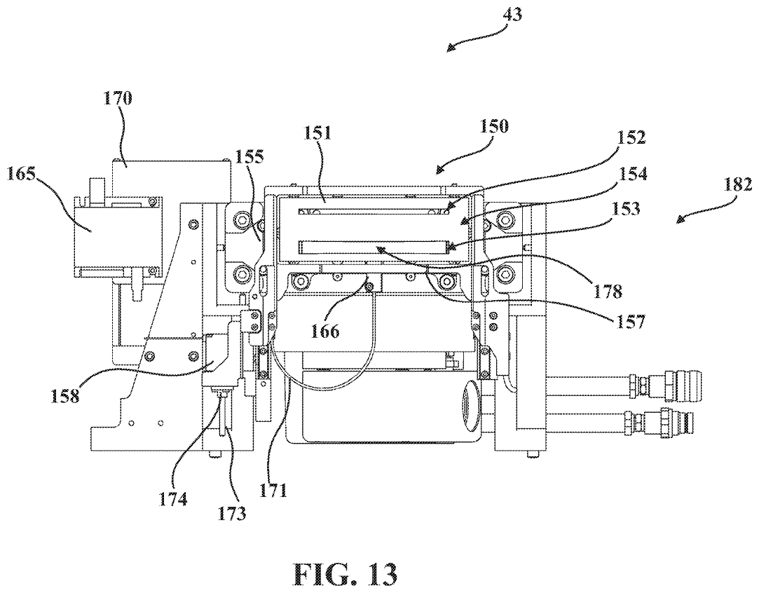

FIGS. 13, 14, and 15 illustrate the adjustable collimator assembly 150 moving between positions 180, 181, 182. FIG. 13 illustrates the adjustable collimator assembly 150 in position 182 with aperture 153 aligned with the x-ray beam outlet port 178. As described above. in position 182, the x-ray beam is not blocked by the limiter 151 between the beam reducer 162 and the x-ray detector 45. FIG. 14 illustrates the adjustable collimator assembly 150 in position 180 which positions the solid portion/blocker 154 of the limiter 151 in line with the x-ray beam outlet port 178. In some examples, a third transmission parameter, such as a third x-ray beam intensity that is less than the first x-ray beam intensity and the second x-ray beam intensity, corresponds with the alignment of the blocker 154 of the limiter 151 with the x-ray beam outlet port 178 in position 180. In this position 180, the x-ray beam is unable to travel past the limiter 151 to the x-ray detector 45 since the pathway is blocked by the limiter 151. In some examples, the imaging system 100 may be in a non-scanning mode. Similar to FIG. 12, FIG. 15 illustrates the adjustable collimator assembly 150 in position 181 which positions the limiter 151 such that aperture 152 is aligned with the x-ray beam outlet port 178, reducing the x-ray beam between the beam reducer 162 and the x-ray detector 45 when the imaging system is in a scout scan mode.

As is illustrated in FIGS. 7-15, the reference detector 166 may be positioned proximate to an edge of the x-ray beam outlet port, such that the reference detector 166 does not cast a “shadow” on the object being imaged. The reference detector 166 may be positioned behind a collimator 168 so that it may measure the flux of the x-ray photons prior to the photons being collimated. As shown in FIGS. 7A-7B and 8-15, the reference detector 166 is provided at the x-ray source 43 to measure the flux of the photons leaving the x-ray tube before the photons impinge on the object being imaged. The reference detector 166 may be a single x-ray sensitive element (e.g., a scintillator, such as a cadmium tungstate crystal), and may be identical to the x-ray sensitive elements in each of the detector elements of the detector system 45. A fiber optic cable 171 may be coupled to the reference detector 166 to transmit an optical signal from the reference detector 166 to a reference detector module 170. The reference detector module 170 may be located in a temperature-controlled location on the rotor 41 (e.g., in a location where heat from the x-ray source 43 does not interfere with operation of components, such as a photodiode, of the reference detector module 170). The reference detector 166, fiber optic cable 171 and reference detector module 170 may be potted (e.g., with carbon-filled epoxy) to prevent unwanted light from contaminating the optical signal. The reference detector module 170 may include a photodiode that generates an electronic signal in response to the incident optical signal from the reference detector 166, and associated electronics (e.g., A/D converter, FPGA, etc.) that may convert the electronic signal into a digital signal that may be fed to the processor 102 for use in performing the tomographic reconstruction. The signal from the reference detector 166 may be sent in a digital video format, such as Camera Link. In examples, the digital reference detector signal from the reference detector module 170 may be sent to the detector 45, where the signal may be embedded within the digital image data from the detector modules 107 before it is transmitted to the processor 102 for reconstruction. For example, the signal from the reference detector 166 may be sent to the headboard of the detector 45. The headboard may then send the signal to the first detector module 107, such as with its clock signal, and the reference detector signal may be appended to the digital image data from the first detector module 107 when it is transmitted to the next module 107 along the line. The signal from the reference detector 166 may thus propagate down the line of detector modules 107 in a daisy-chain fashion and may then be fed to the processor 102 for tomographic reconstruction.

The reference detector 166 and fiber optic cable 171 assembly according to one examples. The reference detector 166 may be embedded in a housing, which may be a brass housing having a hole for x-ray photons to enter. An RTD may also be provided in the housing. The fiber optic cable 171 may have a polished first end that is bonded to a polished end of the reference detector 166 (e.g., scintillator crystal) for receiving incident light from the reference detector 166. The subassembly of reference detector 166 and fiber optic cable 171 may inserted into the housing (along with the RTD) and potted within the housing, which may be a brass housing. The fiber optic cable 171 may have a polished second end that may be bonded to a photodiode. One or more wire leads may couple the RTD output to an electronics module (e.g., circuit board).

The reference detector 166 may also include a temperature sensor, such as a resistance temperature detector (RTD) that may generate an electronic signal indicative of the temperature within the x-ray source 43. The temperature signal may be a digital signal that may be embedded within the image data stream that is sent to the processor 102 for tomographic reconstruction in the manner described above for the reference detector signal.

As noted above, the x-ray imaging device 10 includes the x-ray source 43, such as an x-ray tube, that is configured to direct radiation, including collimated x-ray radiation, onto the x-ray detector 45. The x-ray source 43 may include a beam steering mechanism that may alter the direction of the output beam by a particular angle, such as 90° or more. In some examples, the x-ray imaging device 10 may include two or more radiation sources and two or more detectors such that at least a portion of the output radiation beam is alternately centered on a first detector and a second detector, which may be spaced by 90° to provide bi-planar imaging, such as described in U.S. Pat. No. 9,526,461 filed Jun. 25, 2013, the entire disclosure of which is hereby incorporated by reference.

In this application, including the definitions below, the term “controller” may be replaced with the term “circuit.” The term “controller” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The one or more controller(s) may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4.

The one or more controllers may communicate with other controllers using the interface circuit(s). Although the controller may be depicted in the present disclosure as logically communicating directly with other controllers, in various configurations the controller may actually communicate via a communications system. The communications system includes physical and/or virtual networking equipment such as hubs, switches, routers, and gateways. In some configurations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various configurations, the functionality of the controller may be distributed among multiple controllers that are connected via the communications system. For example, multiple controllers may implement the same functionality distributed by a load balancing system. In a further example, the functionality of the controller may be split between a server (also known as remote, or cloud) controller and a client (or, user) controller.

Some or all hardware features of a controller may be defined using a language for hardware description, such as IEEE Standard 1364-2005 (commonly called “Verilog”) and IEEE Standard 10182-2008 (commonly called “VHDL”). The hardware description language may be used to manufacture and/or program a hardware circuit. In some configurations, some or all features of a controller may be defined by a language, such as IEEE 1666-2005 (commonly called “SystemC”), that encompasses both code, as described below, and hardware description.

The various controller programs may be stored on a memory circuit. The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter. (v) source code for compilation and execution by a just-in-time compiler, etc. As examples only, source code may be written using syntax from languages including C, C++, C #, Objective C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SENSORLINK, and Python®.

Several examples have been discussed in the foregoing description. However, the examples discussed herein are not intended to be exhaustive or limit the disclosure to any particular form. The terminology that has been used is intended to be in the nature of words of description rather than of limitation. Many modifications and variations are possible in light of the above disclosure and the disclosure may be practiced otherwise than as specifically described.

The present disclosure also comprises the following clauses, with specific features laid out in dependent clauses, that may specifically be implemented as described in greater detail with reference to the configurations and drawings above.

### Clauses

I. An imaging system comprising:
a base;
a gantry connected with the base, the gantry including:
   an x-ray source to produce an x-ray beam,
   an x-ray detector to receive the x-ray beam from the x-ray source,
   an adjustable collimator including a limiter disposed between the x-ray source and the x-ray detector and arranged for movement between:
      a first position to at least partially limit transmission of the x-ray beam towards the x-ray detector according to a first transmission parameter, and
      a second position to at least partially limit transmission of the x-ray beam towards the x-ray detector according to a second transmission parameter different from the first transmission parameter; and
a control system with a controller configured to move the limiter of the adjustable collimator between the first position and the second position to adjust transmission of the x-ray beam from the x-ray source towards the x-ray detector.

II. The imaging system of clause I, wherein the limiter defines a first aperture arranged to limit transition of the x-ray beam towards the x-ray detector according to the first transmission parameter during operation of the adjustable collimator with the limiter in the first position.

III. The imaging system of clause II, wherein the limiter includes a blocker arranged to substantially inhibit transmission of the x-ray beam towards the x-ray detector according to the second transmission parameter during operation of the adjustable collimator with the limiter in the second position.

IV. The imaging system of any of clauses II-III, wherein the limiter defines a second aperture arranged to limit transmission of the x-ray beam towards the x-ray detector according to the second transmission parameter during operation of the adjustable collimator with the limiter in the second position.

V. The imaging system of clause IV, wherein the second aperture is at least partially smaller than the first aperture such that transmission of the x-ray beam towards the x-ray detector during operation of the adjustable collimator with the limiter in the second position is limited more than during operation of adjustable collimator with the limiter in the first position.

VI. The imaging system of clause V, wherein the control system is disposed in communication with the x-ray source, the x-ray detector, and the controller connected with the adjustable collimator; and
wherein the control system selectively operable between a first imaging mode and a second imaging mode different from the second imaging mode.

VII. The imaging system of clause VI, wherein the first imaging mode is further defined as a helical scan mode and the second imaging mode is further defined as a scout scan mode.

VIII. The imaging system of clause VII, wherein the limiter of the adjustable collimator is arranged:
in the first position during operation in the helical scan mode, and
in the second position during operation in the scout scan mode.

IX. The imaging system of any of clauses IV-VIII, wherein the limiter of the adjustable collimator is further arranged for movement to a third position to at least partially limit transmission of the x-ray beam towards the x-ray detector according to a third transmission parameter different from the first transmission parameter and from the second transmission parameter.

X. The imaging system of clause IX, wherein the limiter includes a blocker arranged to substantially inhibit transmission of the x-ray beam towards the x-ray detector according to the third transmission parameter during operation of the adjustable collimator with the limiter in the third position.

XI. The imaging system of any of clauses I-X, wherein the adjustable collimator further includes a limiter mount supporting the limiter for movement, and a limiter actuator disposed in communication with the controller and operatively attached to the limiter and the limiter mount to move the limiter relative to the limiter mount between the first position and the second position.

XII. The imaging system of clause XI, wherein the limiter of the adjustable collimator is further arranged for movement to a third position to at least partially limit transmission of the x-ray beam towards the x-ray detector according to a third transmission parameter different from the first transmission parameter and from the second transmission parameter; and
wherein the limiter actuator is further configured to move the limiter relative to the limiter mount between the first position, the second position, and the third position.

XIII. The imaging system of clause XII, wherein the limiter defines a first aperture arranged to limit transition of the x-ray beam towards the x-ray detector according to the first transmission parameter during operation of the adjustable collimator with the limiter in the first position;
wherein the limiter includes a blocker arranged to substantially inhibit transmission of the x-ray beam towards the x-ray detector according to the second transmission parameter during operation of the adjustable collimator with the limiter in the second position; and
wherein the limiter defines a second aperture arranged to limit transmission of the x-ray beam towards the x-ray detector according to the second transmission parameter during operation of the adjustable collimator with the limiter in the second position.

XIV. The imaging system of any of clauses I-XIII, wherein the limiter is at least partially formed from tungsten.

XV. The imaging system of any of clauses I-XIV, further comprising a non-adjustable collimator located between the x-ray source and the adjustable collimator.

XVI. A method of adjusting a transmission parameter of an imaging system, the imaging system including: a base; a gantry connected with the base, the gantry including: an x-ray source to produce an x-ray beam; an x-ray detector to receive the x-ray beam from the x-ray source; an adjustable collimator including a limiter disposed between the x-ray source and the x-ray detector and arranged for movement between a first position and a second position; and a control system with a controller configured to move the limiter of the adjustable collimator between the first position and the second position, the method comprising:
controlling the control system to select a scan mode of the imaging system;
moving the limiter to a position corresponding with the scan mode of the imaging system; and
transmitting an x-ray beam from the x-ray source through the limiter and towards the x-ray detector.

XVII. The method of clause XVI, wherein controlling the control system to select a scan mode of the imaging system includes controlling the control system to select a helical scan mode.

XVIII. The method of clause XVII, wherein transmitting the x-ray beam from the x-ray source through the limiter and towards the x-ray detector includes transmitting the x-ray beam from the x-ray source through the limiter and towards the x-ray detector with the limiter of the adjustable collimator in a first position to at least partially limit transmission of the x-ray beam towards the x-ray detector according to a first transmission parameter.

XIX. The method of clause XVIII, wherein controlling the control system to select a scan mode of the imaging system includes controlling the control system to select a scout scan mode.

XX. The method of clause XIX, wherein transmitting the x-ray beam from the x-ray source through the limiter and towards the x-ray detector includes transmitting the x-ray beam from the x-ray source through the limiter and towards the x-ray detector with the limiter of the adjustable collimator in a second position to at least partially limit transmission of the x-ray beam towards the x-ray detector according to a second transmission parameter different from the first transmission parameter such that transmission of the x-ray beam towards the x-ray detector during operation of the adjustable collimator with the limiter in the second position is limited more than during operation of adjustable collimator with the limiter in the first position.

What is claimed is:

1. An imaging system comprising:
a base;
a gantry connected with the base, the gantry including:
an x-ray source to produce an x-ray beam,
an x-ray detector to receive the x-ray beam from the x-ray source,
an adjustable collimator including a limiter disposed between the x-ray source and
the x-ray detector and arranged for movement between:
a first position to at least partially limit transmission of the x-ray beam towards the x-ray detector according to a first transmission parameter, and
a second position to at least partially limit transmission of the x-ray beam towards the x-ray detector according to a second transmission parameter different from the first transmission parameter; and
a control system with a controller configured to move the limiter of the adjustable collimator between the first position and the second position to adjust transmission of the x-ray beam from the x-ray source towards the x-ray detector.

2. The imaging system of claim 1, wherein the limiter defines a first aperture arranged to limit transition of the x-ray beam towards the x-ray detector according to the first transmission parameter during operation of the adjustable collimator with the limiter in the first position.

3. The imaging system of claim 2, wherein the limiter includes a blocker arranged to substantially inhibit transmission of the x-ray beam towards the x-ray detector according to the second transmission parameter during operation of the adjustable collimator with the limiter in the second position.

4. The imaging system of claim 2, wherein the limiter defines a second aperture arranged to limit transmission of the x-ray beam towards the x-ray detector according to the second transmission parameter during operation of the adjustable collimator with the limiter in the second position.

5. The imaging system of claim 4, wherein the second aperture is at least partially smaller than the first aperture such that transmission of the x-ray beam towards the x-ray detector during operation of the adjustable collimator with the limiter in the second position is limited more than during operation of adjustable collimator with the limiter in the first position.

6. The imaging system of claim 5, wherein the control system is disposed in communication with the x-ray source, the x-ray detector, and the controller connected with the adjustable collimator; and
wherein the control system selectively operable between a first imaging mode and a second imaging mode different from the second imaging mode.

7. The imaging system of claim 6, wherein the first imaging mode is further defined as a helical scan mode and the second imaging mode is further defined as a scout scan mode.

8. The imaging system of claim 7, wherein the limiter of the adjustable collimator is arranged:
in the first position during operation in the helical scan mode, and
in the second position during operation in the scout scan mode.

9. The imaging system of claim 4, wherein the limiter of the adjustable collimator is further arranged for movement to a third position to at least partially limit transmission of the x-ray beam towards the x-ray detector according to a third transmission parameter different from the first transmission parameter and from the second transmission parameter.

10. The imaging system of claim 9, wherein the limiter includes a blocker arranged to substantially inhibit transmission of the x-ray beam towards the x-ray detector according to the third transmission parameter during operation of the adjustable collimator with the limiter in the third position.

11. The imaging system of claim 1, wherein the adjustable collimator further includes a limiter mount supporting the limiter for movement, and a limiter actuator disposed in communication with the controller and operatively attached to the limiter and the limiter mount to move the limiter relative to the limiter mount between the first position and the second position.

12. The imaging system of claim 11, wherein the limiter of the adjustable collimator is further arranged for movement to a third position to at least partially limit transmission of the x-ray beam towards the x-ray detector according to a third transmission parameter different from the first transmission parameter and from the second transmission parameter; and
wherein the limiter actuator is further configured to move the limiter relative to the limiter mount between the first position, the second position, and the third position.

13. The imaging system of claim 12, wherein the limiter defines a first aperture arranged to limit transition of the x-ray beam towards the x-ray detector according to the first transmission parameter during operation of the adjustable collimator with the limiter in the first position;
wherein the limiter includes a blocker arranged to substantially inhibit transmission of the x-ray beam towards the x-ray detector according to the second transmission parameter during operation of the adjustable collimator with the limiter in the second position; and
wherein the limiter defines a second aperture arranged to limit transmission of the x-ray beam towards the x-ray detector according to the second transmission parameter during operation of the adjustable collimator with the limiter in the second position.

14. The imaging system of claim 1, wherein the limiter is at least partially formed from tungsten.

15. The imaging system of claim 1, further comprising a non-adjustable collimator located between the x-ray source and the adjustable collimator.

16. A method of adjusting a transmission parameter of an imaging system, the imaging system including: a base; a gantry connected with the base, the gantry including: an x-ray source to produce an x-ray beam; an x-ray detector to receive the x-ray beam from the x-ray source; an adjustable collimator including a limiter disposed between the x-ray source and the x-ray detector and arranged for movement between a first position and a second position; and a control system with a controller configured to move the limiter of the adjustable collimator between the first position and the second position, the method comprising:
controlling the control system to select a scan mode of the imaging system;
moving the limiter to a position corresponding with the scan mode of the imaging system; and
transmitting an x-ray beam from the x-ray source through the limiter and towards the x-ray detector.

17. The method of claim 16, wherein controlling the control system to select a scan mode of the imaging system includes controlling the control system to select a helical scan mode.

18. The method of claim 17, wherein transmitting the x-ray beam from the x-ray source through the limiter and towards the x-ray detector includes transmitting the x-ray beam from the x-ray source through the limiter and towards the x-ray detector with the limiter of the adjustable collimator in a first position to at least partially limit transmission of the x-ray beam towards the x-ray detector according to a first transmission parameter.

19. The method of claim 18, wherein controlling the control system to select a scan mode of the imaging system includes controlling the control system to select a scout scan mode.

20. The method of claim 19, wherein transmitting the x-ray beam from the x-ray source through the limiter and towards the x-ray detector includes transmitting the x-ray beam from the x-ray source through the limiter and towards the x-ray detector with the limiter of the adjustable collimator in a second position to at least partially limit transmission of the x-ray beam towards the x-ray detector according to a second transmission parameter different from the first transmission parameter such that transmission of the x-ray beam towards the x-ray detector during operation of the adjustable collimator with the limiter in the second position is limited more than during operation of adjustable collimator with the limiter in the first position.
